(19) United States
(12) Patent Application Publication
(10) Pub. No.: US 2002/0098501 A1
(43) Pub. Date: Jul. 25, 2002

(54) Method for identifying bacteremia associated with community acquired pneumonia

(76) Inventors: Richard Glenn Wunderink, Germantown, TN (US); Grant William Waterer, Yokine (AU)

Correspondence Address: Licata & Tyrrell P.C., 66 E. Main Street, Marlton, NJ, 08053, US

(21) Appl. No.: 09/974,392

(22) Filed: Oct. 10, 2001

Related U.S. Application Data
(60) Provisional application No. 60/238,975, filed on Oct. 10, 2000.

(51) Int. Cl. C12Q 1/68

(52) U.S. Cl. 435/6

(57) ABSTRACT

A method of diagnosing a disease associated with a genetic polymorphism in a FcgRII gene comprises determining the genotype of said FcgRII gene in an animal. The method can be used to identify predisposition or susceptibility to bacteremia in patients when they have CAP. Compositions for said diagnosis are provided. Methods of treatment of such patients are provided.

### INTRODUCTION

This application claims the benefit of priority from U.S. provisional application Serial No. 60/238,975 filed Oct. 10, 2000.

### FIELD OF THE INVENTION

This invention relates to methods for identifying predisposition or susceptibility to bacteremia in patients having Community-Acquired Pneumonia (CAP), by screening for the presence of a risk polymorphism of FcγRIIa, an IgG receptor. FcγRIIa (or FcgRIIa) is widely described in the literature including Osborne et al. J. Immunol. Methods, 1994; 173:207-217. More specifically, this invention relates to carriage of the guanine (G) allele at +494 of the FcyRIIa gene being associated with an increased risk of bacteremia in patients when they have CAP. The invention also relates to compositions for screening for the polymorphism and improved treatment choices for patients diagnosed as being susceptible to bacteremia by the methods of the present invention.

### BACKGROUND OF THE INVENTION

Pneumonia is a common clinical entity, particularly among the elderly. A thorough understanding of the epidemiology and microbiology of community-acquired pneumonia (CAP) is essential for appropriate diagnosis and management. Although the microbiology of CAP has remained relatively stable over the last decade, there is new information on the incidence of atypical pathogens, particularly in patients not admitted to hospital, and new information on the incidence of pathogens in cases of severe CAP and in CAP in the elderly. Recent studies have provided new data on risk factors for mortality in CAP, which can assist the clinician in decisions about the need for hospital admission. The emergence of antimicrobial resistance in Streptococcus, the organism responsible for most cases of CAP, has greatly affected the approach to therapy, especially in those patients who are treated empirically. Guidelines for the therapy of CAP have been published by the American Thoracic Society, the British Thoracic Society, and, most recently, the Infectious Diseases Society of America and others. These guidelines differ in their emphasis on empirical versus pathogenic-specific management.

CAP remains a significant health problem and patients continue to die despite receiving appropriate antibiotic therapy. Modification of the host immune response, both anti- and pro-inflammatory approaches, has yet to live up to the promise of improved outcome. Despite this, there is significant reason for optimism. Some immunomodulatory therapies clearly have efficacy in some patients. As the understanding of the immune response to pneumonia improves, the ability to tailor specific therapies for individual patients will also improve, hopefully avoiding the deleterious effects that have so far prevented the development of an effective immune based therapy. The possibility of delivering cytokines directly to the lung, is a particularly promising way of achieving the desired pulmonary effect without systemic side effects. Corticosteroids are currently unique in that they have a proven role in the therapy of pneumonia due to P. carinii. The development of pathogen specific therapies, such as INF for L. pneumophila, based on an improved understanding of host-pathogen interactions, are awaited.

The past 20 years has seen an explosion in our knowledge of human immunology and we are only now beginning to explore the therapeutic possibilities this has made available. The next 10 years promises to finally provide a significant advance in the therapy of pneumonia, the first substantial gain since penicillin.

In light of the prevalence of CAP and the evolution of resistance in the most common bacterial CAP pathogen, physicians advise obtaining specimens for culture of CAP pathogens and analyzing patterns of susceptibility, especially of S. pneumonia, in their communities, using antibiotics appropriately and prudently, according to prevailing susceptibilities when empirical treatment is called for, and immunizing their susceptible patients with pneumococcal and influenza vaccines. This is because the mortality of patients with severe CAP approaches or may exceed 20%, compared to less than 1% for patients with non-severe CAP (Fine et al. New Engl. J. Med. 1997.336:243-250, British Thoracic Society, Q. J. Med. 1987.239:192-220, Niederman et al. Am. Rev. Resp. Dis. 1993.148:1418-1426).

Phagocytosis of bacteria, especially encapsulated microorganisms, by neutrophils is an important defense mechanism in animals. A polymorphism of FcgammaRIIa, an IgG receptor, is associated with impaired phagocytosis of bacteria. Others have suggested it is associated with increased bacteremia in pneumococcal and meningococcal disease (Platonov et al. Clin. Infect. Dis. 1998.27:746-750, Yee et al. Clin. Infect. Dis. 2000.30:25-28). It has now surprisingly been found that carriage of the FcgammaRIIa G allele is associated with an increased risk of bacteremia in patients having CAP. In such cases an ability to identify predisposition or susceptibility to bacteremia in patients having CAP would be of distinct advantage and may lead to improved outcomes and lower medical costs for such patients as bacteremia is widely known to be a risk factor for poor outcome in CAP patients.

### BRIEF SUMMARY OF THE INVENTION

It is an object of the invention to provide methods of identifying a predisposition or susceptibility to bacteremia in patients having CAP. A further object is to provide, following such identification, methods of identifying patients for alternative management of CAP before the disease becomes significantly established. Thus, the invention also relates to carriage of the guanine (G) allele of the FcgammaRIIa gene being associated with an increased risk of bacteremia in patients when they have CAP and improved treatment choices for patients identified as having a predisposition or susceptibility to bacteremia when they have CAP by the method of the present invention. The invention also relates to compositions for screening for the polymorphism and kits for such screening and methods of treatment of such patients.

Other preferred embodiments of the present invention will be apparent to one of ordinary skill in light of the following description of the invention and of the claims.

### DETAILED DESCRIPTION OF THE INVENTION

The first aspect of the invention provides a method of identifying an animal, including a human, predisposed or susceptible to a disease associated with a genetic polymorphism in a FcgRII gene, said method comprising determining the genotype of said FcgRII gene in said animal. In an embodiment of the invention, the method of diagnosis is to screen for an individual at risk of a condition or disease such as predisposition or susceptibility to bacteremia when they have CAP correlated with a FcgRII gene polymorphism (the G allele) at +494.

The invention is based upon the observation reported herein of a correlation between polymorphisms in the FcgRII gene, specifically (the G allele) at position +494, and predisposition or susceptibility to bacteremia in patients when they have CAP. The invention is of advantage in that by screening for the presence of the polymorphism it is possible to identify individuals likely to have a genetic predisposition or susceptibility to the disease. It may also result in substantially different management, both prevention and treatment, if CAP occurs, with subsequent substantial improvement in mortality and morbidity from such patients.

In an embodiment of the invention identification is carried out by determining whether a FcgRII gene contains a polymorphism G at +494. Possessing a fragment that contains the G at +494 correlates with increased risk of predisposition or susceptibility to bacteremia in patients when they have CAP. As a human genome contains two FcgRII genes, one on each of a pair of chromosomes, an individual can accordingly be found to be homozygous or heterozygous for the risk polymorphism, or to lack the risk polymorphism.

Genotypic and allelic frequencies of this invention are readily determined by a number of methods known to those skilled in the art. Examples used in the present invention are shown in the Example below and include using PCR amplification and dot blot analysis.

The method conveniently comprises amplifying a fragment of a FcgRII gene to produce copies and determining whether copies of the fragment contain the G allele.

Another suitable technique is to amplify the fragment using PCR techniques, producing copies of a fragment that is at least 500 base pairs in length, preferably at least 600 base pairs in length. It is preferred that the PCR primers are selected so as to amplify a region of the gene that is about 740 base pairs in length. PCR techniques are well known in the art and it would be within the ambit of a person of ordinary skill in this art to identify primers for amplifying a suitable section of the applicable exon of the FcgRII gene. PCR techniques are described for example in EP-A-0200362 and EP-A-0201 184. In a further embodiment of the invention, the diagnostic method comprises analysis of the FcgRII gene using single strand conformational polymorphism (SSCP) mapping to determine whether the FcgRII gene is the risk or the non-risk form, (the G allele).

As described above, in preferred embodiments of the first aspect of the invention, the method comprises screening a FcgRII gene, and this screening is conveniently carried out by any one of a number of suitable techniques that are known in the art, and may be conveniently selected from amplification of a nucleic acid sequence located within the FcgRII gene, Southern blotting of regions of the gene and single strand conformational polymorphism mapping of regions within the gene or as described in the example below. The genotype in that region is also optionally determined using hybridization probes adapted selectively to hybridize with the particular polymorphism of the FcgRII gene at the +494 location which is associated with predisposition or susceptibility to disease. A probe used for hybridization detection methods must be in some way labeled so as to enable detection of successfully hybridization events. This is optionally achieved by in vitro methods such as nick-translation, replacing nucleotides in the probe by radioactively labeled nucleotides, or by random primer extension, in which non-labeled molecules act as a template for the synthesis of labeled copies. Other standard method of labeling probes so as to detect hybridization are known to those skilled in this art.

According to a second aspect of the invention there is provided a method of diagnosis and therapy comprising identifying a predisposition or susceptibility to bacteremia in patients when they have CAP according to the method of the first aspect of the invention and treating an individual having such increased risk by methods known to those of skill in the art and by using the novel treatment and prophylactic methods described below. It may be preferable to do so prior to the patient having CAP. CAP can be diagnosed by methods known to those of skill in the art and as described herein.

Known therapies for such CAP patients with predisposition or susceptibility to bacteremia can be effective in halting advancement of the disease, or at least slowing the advancement. FcgRII gene analysis may also lead to more appropriate placement of patients into intensive care/critical care units, an important factor in optimizing survival from CAP in such patients. It is thus an advantage of the invention that by early identification of such patients, outcome in CAP is improved. Preventative therapy can be started as soon as possible, optimizing any interventions potential (such as vaccination or immunomodulatory therapy) for affecting outcome. As alternative diagnostic methods improve and are developed, so the invention can add to the total knowledge of the risk of developing bacteremia of an individual having CAP. The decision of a physician on how and whether to initiate therapy in anticipation of the disease can be taken with increased confidence.

A variety of suitable treatments of CAP patients with bacteremia are described in the art and herein. See also, Hirani and MacFarlane Thorax 1997.52:17-21, Pachon J. et al. Am. Rev. Resp. Dis. 1990.142:369-373, Ruiz M. et al. Am. J Respir. Crit. Care. Med. 1999.160:923-929, Leeper and Torres Clin. Chest. Med. 1995.16:155-171. Other treatments will be known to persons of skill in the art.

Another aspect of the invention also provides a composition for use in identifying an animal predisposed or susceptible to a disease associated with a genetic polymorphism in a FcgRII gene, said compositions comprising one or more primer nucleic acid molecules adapted to amplify a portion of the FcgRII gene selected from a portion of the gene around the +494 location.

The composition of the third aspect of the invention may comprise a nucleic acid molecule capable of identifying the +494 polymorphism (G allele) in said FcgRII gene, said polymorphism being indicative of a risk genotype in said animal.

A further embodiment of the third aspect of the invention provides a composition for identifying predisposition or susceptibility to bacteremia in patients when they have CAP, comprising means for determining genotype of a FcgRII gene of an individual, for example whether an individual is homozygous or heterozygous for polymorphic variants of a FcgRII gene at the +494 location such as the method provided in the example herein or other methods known to those of skill in the art.

In an embodiment of the invention, a composition comprises PCR primers adapted to amplify a DNA sequence within and around the FcgRII gene +494 polymorph location, wherein alternative versions of the gene are distinguished one from another.

In a further aspect of the invention there is provided a kit comprising a composition as described above and an indicator composition for indicating how possessing a polymorphic version of a FcgRII gene correlates with the presence of predisposition or susceptibility to bacteremia in patients when they have CAP.

Diagnostic kits are typically accompanied by or comprise a chart or other visual aid for assistance in interpreting the results obtained using the kit. Suitable indicator compositions for use in the diagnostic kit of the invention include a leaflet or other visual reminder that possessing the risk polymorphism version of a FcgRII gene (G allele) correlates with the increased risk of predisposition or susceptibility to bacteremia in patients when they have CAP.

In a still further aspect of the invention there is provided use, in the manufacture of means for identifying whether an individual has a predisposition or susceptibility to bacteremia when they contract CAP, of PCR primers adapted to amplify a region around +494 in the FcgRII gene. Alternative versions of the gene are typically distinguished one from another by means known to those skilled in the art. Multiple techniques exists and are known to one skilled in the art in the manufacture of means for diagnosing whether an individual has a predisposition or susceptibility to bacteremia when they contract CAP by determining the G allele of the gene herein, for example by PCR primers adapted to amplify a region around +494 in the FcgRII gene. The presence of a G or A allele can then determined by dot blot analysis (see Osborne et al. J. Immunol. Methods. 1994.173:207-217). One could use restriction analysis which generates different fragment lengths for the G allele, identified by electrophoresis on an agarose gel where the different fragments migrate different amounts based on their size.

According to the invention, an individual who is homozygous for a risk polymorphism, that is to say homozygous for carriage of the G allele at FcgRIIa+494 (GG), is classified as being at highest risk. An individual being heterozygous (GA) is classified as having moderate risk.

Optionally, the assessment of an individual's risk factor according to any aspect of the invention is calculated by determining the genotype of a FcgRII gene polymorphism and combining the result with analysis of other known genetic or physiological or dietary or other risk factors known to those of skill in the art. The invention in this way provides further information on which measurement of an individual's risk can be based.

In another embodiment of the invention, the results of the genotyping done herein are used, along with other measures and disease parameters, by treatment providers to determine the best course of treatment for the patient having been determined as susceptible to bacteremia in CAP by the methods determined as susceptible to bacteremia in CAP by the methods of this invention.

This invention provides methods of treating abnormal conditions related to reduced IgG2 binding capacity of the Fcgamma receptor when the G allele is present. The mutation causes an amino acid change in the Fcgamma receptor—arginine to histidine, such that the affinity for the receptor for IgG2 is higher when the A allele (histidine amino acid substitution) is present, hence phagocytic cells are more effective at removing bacteria.

Therefore, methods of treating such at risk patients of this invention are provided by administering an effective amount of compounds in pharmaceutically acceptable carriers to such patients to stimulate phagocytosis in such patients who have been identified as predisposed or susceptible to bacteremia when they have CAP (i.e., with reduced phagocytic capacity due to the detrimental polymorphism (G allele at site +494)) by the method of this invention.

In still another aspect, expression of the gene encoding Fcgamma receptor (G allele at +494) polypeptide can be inhibited using expression blocking techniques. Known such techniques involve the use of antisense sequences, either internally generated or separately administered. See, for example, O'Connor, J. Neurochem. 1991.56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al. Nucleic Acids Res. 1979.6:3073; Cooney et al. Science 1988.241:456; Dervan et al. Science 1991.251:1360. These oligomers can be administered per se or the relevant oligomers can be expressed in vivo.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following example, which is included herewith for purposes of illustration only and is not intended to be limiting of the invention.

### EXAMPLE

1

Methods:

Subjects were recruited as part of a prospective cohort study of patients with CAP. Only patients who had at least 2 blood cultures prior to the administration of antibiotics were considered eligible for this analysis. Genotype was determined using PCR amplification and dot blot analysis. The significance of trends was assessed using Fishers-exact test.

Results:

163 patients met the criteria for review; 27 had positive blood cultures. The number of patients in each genotype were AA—26(16%), GA—89(55%), GG—48 (29%). The risk of bacteremia in each genotype was AA—0%, GA—24%, GG—12.5% (p=0.03 AA vs non AA, p=0.2 GA vs GG). Logistic regression analysis showed the protective effect of the AA genotype to be independent of age, sex, race and alcohol consumption (p=0.001).

Conclusion:

Carriage of the G allele of the FcγRIIa gene is associated with a significantly greater risk of bacteremia in patients when they have CAP.

What is claimed is:

1. A method of identifying an animal predisposed or susceptible to bacteremia when they have CAP associated with a genetic polymorphism in a FcgRII gene at the +494 locus, said method comprising determining the genotype of said FcgRII gene in said animal, and identifying said animal based on said genotype.

2. The method of claim 1 further comprising determining whether said animal is homozygous or heterozygous for the G allele of a FcgRII gene at the +494 locus.

3. A method of identifying an individual having a predisposition or susceptibility to bacteremia when they have CAP by determining whether the individual possesses a polymorphic risk version of a FcgRII gene at the +494 locus, a polymorphic risk version of the gene being one that has an G at site +494, the method comprising:
(a) using dot blot analysis of a portion of the FcGammaRII gene amplified by polymerase chain reaction to determine if a G is present at the +494 site and not A,
(b) testing whether the copies contain an A or a G at site +494 through known differences in how such fragments appear on dot blot analysis, and thereby determining whether the individual is homozygous or heterozygous for a polymorphic risk version of the gene, and
(c) identifying an individual predisposed or susceptible to bacteremia when they have CAP as greatest if that individual is homozygous for the polymorphic risk version of the gene at the +494 site (GG), moderate if that individual is heterozygous for the polymorphic risk version at the +494 site (GA), and least if that individual lacks the polymorphic risk version at the +494 site (AA).

4. A method of managing and treating patients with a predisposition to or who are susceptible to bacteremia when they have CAP comprising, determining whether the individual possesses a polymorphic risk version of a FcgRII gene at the +494 locus, a polymorphic risk version of the gene being one that has a G at site +494, wherein the management and treatment of such patient having such polymorphism are promptly treated and managed as if such patient is predisposed or susceptible to bacteremia when they have CAP.
